(19) United States
(12) Patent Application Publication
(10) Pub. No.: US 2011/0076315 A1
(43) Pub. Date: Mar. 31, 2011

(54) Grafts and Stents Having Inorganic Bio-Compatible Calcium Salt

(75) Inventors: R. Michael Casanova, Scottsdale, AZ (US); Chandrashekhar P. Pathak, Phoenix, AZ (US)

(73) Assignee: C.R Bard, Inc., Murray Hill, NJ (US)

(21) Appl. No.: 11/916,813

(22) PCT Filed: Jun. 8, 2006
(86) PCT No.: PCT/US2006/022359

§ 371 Date: Aug. 27, 2010

Related U.S. Application Data
(60) Provisional application No. 60/689,034, filed on Jun. 8, 2005.

(51) Int. Cl. A61F 2/82; A61K 31/337; A61K 31/436; A61K 33/38; A61P 31/00; A61P 35/00; B82Y 5/00

(52) U.S. Cl. 424/423; 514/449; 514/291; 424/618; 623/1.42; 977/773; 977/810; 977/931

(57) ABSTRACT

The present application discusses techniques and structures that incorporate calcium salts in the luminal surface of grafts. In an embodiment, a graft, stent-graft or TIPS may incorporate bio-compatible calcium salt, which is essentially non-osteoinductive in nature, on the surfaces of the implantable device.

### PRIORITY DATA AND INCORPORATION BY REFERENCE

This application claims priority to U.S. Provisional Patent Application Ser. No. 60/689,034 filed Jun. 8, 2005, entitled “Grafts and Stents Having Inorganic Bio-Compatible Calcium Salt”, which is incorporated herein by reference in its entirety.

### BACKGROUND OF THE INVENTION

Hydroxyapatite Ceramics, specifically Ca₁₀ (PO₄)₆(OH)₂ belong to a large class of calcium phosphate (“CaP”) based bioactive materials used for a variety of biomedical applications, including matrices for drug release control. Other members of the CaP family, such as dicalcium phosphate, e.g., CaHPO₄2H₂O or tricalcium phosphate Ca₃ (PO₄)₂ have also been used for similar purposes. Other forms of Hydroxyapatite (“HA”) are shown and described in U.S. Pat. Nos. 6,730,324 and 6,426,114, which are hereby incorporated by reference as if set forth in their entireties herein. The various forms of the CaP family of materials have been long recognized by those skilled in the art as having highest degree of biocompatibility with human tissue. Examples of prosthetic devices that contemplate the use of, for example, hydroxyapatite, in combination with a prosthetic device include US Patent Application Publication Nos. 20050015154; 20050055097; 20050010297; 20040078090; 20040076656; 20040024456; 20030224032; 20020169465; 20020127261; 20020095157; 2001029382; 20050060020; U.S. Pat. Nos. 5,711,960; and 6,663,664.

Synthetic grafts, including grafts made from polytetrafluoroethylene (“PTFE”), are used in the implantation of grafts in various vessels of the mammalian body such as, for example, vascular (e.g., arterial or venous) and non-vascular ducts (e.g., bile or liver). Examples of ePTFE grafts are shown and described in U.S. Pat. Nos. 5,641,443; 5,827,327; 5,861,026; 6,203,735; 6,221,101; 6,436,135; and 6,589,278, which are hereby incorporated by reference as if set forth in their entireties herein.

Grafts made from materials other than ePTFE include, for example, Dacron mesh reinforced umbilical tissues, bovine collagen, polyester knitted collagen, tricot knitted polyester collagen impregnated, and polyurethane (available under the trademark “Vectra”) have been utilized.

Stent grafts, on the other hand, are prosthetic devices designed to maintain the patency of various vessels in the body, such as the tracheobronchial tree. The device includes a balloon expandable stent encapsulated with ePTFE or a self-expanding Nitinol stent encapsulated with ePTFE and pre-loaded on a flexible delivery system. One example of the latter is known commercially as “Fluency®,” which is marketed by C.R. Bard Peripheral Vascular Inc. Examples of such stent-graft is shown and described in U.S. Pat. Nos. 6,053,941; 6,124,523; 6,383,214; 6,451,047; and 6,797,217, which are hereby incorporated by reference as if set forth in their entireties herein.

### SUMMARY OF THE PRESENT INVENTION

In one aspect, the present invention provides for an implantable graft device that incorporates bio-compatible calcium salt, which is essentially non-osteoinductive in nature, on the either or both of the luminal or abluminal surfaces of the graft device. The graft device includes cardiovascular grafts, vascular (and non-vascular) grafts, vascular or non-vascular stent grafts such as those usable in TIPS procedure.

In another aspect, a graft device is provided. The device includes a layer of synthetic non-metallic material and inorganic bio-compatible calcium salt. The synthetic non-metallic material has a first surface and a second surface spaced apart from the first surface. The inorganic bio-compatible calcium salt is coupled to at least one of the first and second layers of the synthetic non-metallic material.

In yet another aspect, a graft device is provided that includes a stent frame, a synthetic non-metallic material and inorganic bio-compatible calcium salt. The synthetic non metallic material surrounds a portion of the stent frame, and the synthetic non-metallic material has first and second surfaces. The inorganic bio-compatible calcium salt is coupled to at least one of the first and second surfaces of the synthetic non-metallic material.

In a further aspect, a method of endothiealizing a graft is provided. The method can be achieved by coupling ePTFE with inorganic bio-compatible calcium salt to form a composite graft device; and implanting the composite graft device in a mammal.

In yet another aspect, a method of making a composite graft is provided. The method can be achieved by providing a non-metallic material; providing inorganic bio compatible calcium salt; and coupling inorganic bio-compatible calcium salt to the non-metallic material.

### BRIEF DESCRIPTIONS OF THE DRAWINGS

The accompanying drawings, which are incorporated herein and constitute part of this specification, illustrate exemplary embodiments of the invention, and, together with the general description given above and the detailed description given below, serve to explain the features of the invention. It should be understood that the preferred embodiments are not the invention but are some examples of the invention as provided by the appended claims.

FIG. 1 illustrates a cross-section of a preferred graft device.

FIG. 2 illustrates a cross-section of a preferred device used in making the graft device.

FIGS. 3 and 4 illustrate the effect of integration of HA in the material of the graft device.

FIGS. 5A, 5B, 6, 7A, and 7B illustrate various forms of graft device and various views.

FIG. 8A shows a 500× scanning electron micrograph of an inner section of a control graft with no HA.

FIG. 8B shows a 500× scanning electron micrograph of a graft with 10% HA.

FIG. 8C shows a 500× scanning electron micrograph of a graft with 20% HA.

FIG. 8D shows a 500× scanning electron micrograph of a graft with 40% HA.

FIG. 9A shows a 1000× scanning electron micrograph of a longitudinal section of a control graft with no HA.

FIG. 9B shows a 1000× scanning electron micrograph of a longitudinal section of a graft with about 10% HA.

FIG. 9C shows a 1000× scanning electron micrograph of a longitudinal section of a graft with about 20% HA.

FIG. 9D shows a 1000× scanning electron micrograph of a longitudinal section of a graft with about 40% HA.

FIG. 10A shows a 1000× scanning electron micrograph of a radial section of a control graft with no HA.

FIG. 10B shows a 1000× scanning electron micrograph of a radial section of a graft with about 10% HA.

FIG. 10C shows a 1000× scanning electron micrograph of a radial section of a graft with about 20% HA.

FIG. 10D shows a 1000× scanning electron micrograph of a radial section of a graft with about 40% HA.

FIGS. 11A through 11D show EDX graphs of a control graft, a graft with about 10%, 20%, and 40% HA, respectively.

### DETAILED DESCRIPTION OF THE PREFERRED EMBODIMENTS

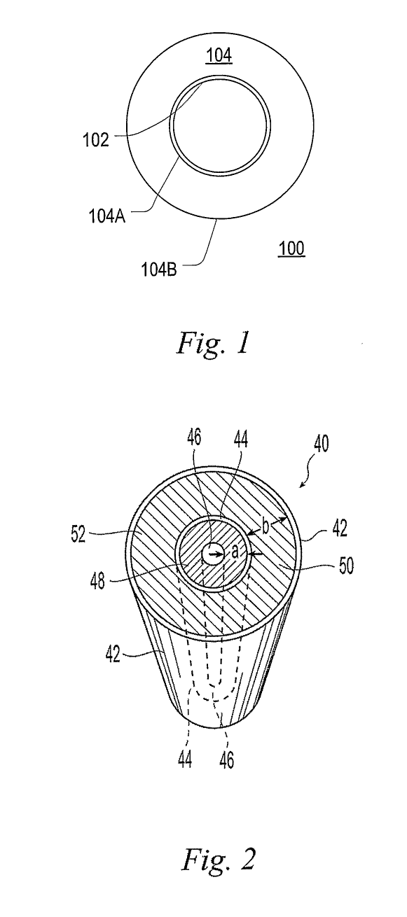

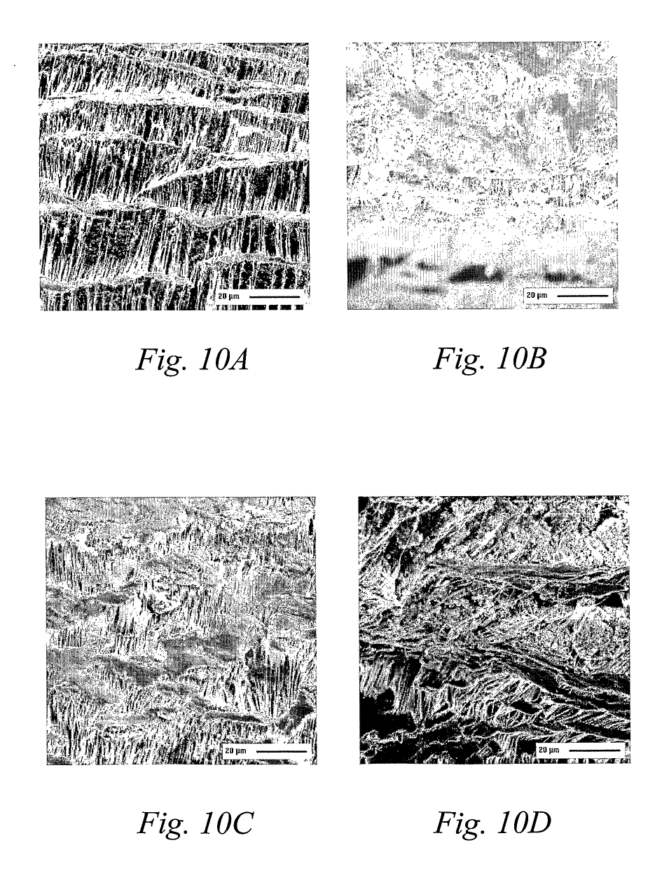

FIGS. 1-11D illustrate the preferred embodiments. As shown in FIG. 1, a cross-section of one of the preferred embodiments of a graft device is shown having a graft device 100 with hydroxyapatite (“HA”) 102 formed on its inner surface 104A (and alternatively, on the outer surface 104B only, both surfaces 104A and 104B, and dispersed or integrated) for graft material 104.

The graft material 104 can be a non-metallic material. Specifically, the non-metallic material can include a synthetic fiber or fabric material such as, for example, Dacron, polyester, PTFE, ePTFE, polyurethane, polyurethane-urea, siloxane, and combinations thereof with an appropriate amount of additives added therein such as, for example, bio-active agents. In the preferred embodiments, the graft material 104 is expanded polytetrafluoroethylene or “ePTFE.”

The ePTFE material for graft 104 can be made by a variety of suitable techniques, one of which is described as follows. A compounding of a polymeric compound is generated by sifting PTFE resin with a suitable amount of lubricant such as, for example, Isopar H, at 30% by weight of the PTFE to enable the PTFE to flow through extrusion equipment. The combined PTFE resin and lubricant are then placed in a shaker device and shaken so that the lubricant coats and penetrates each of the PTFE resin particles. The thoroughly mixed combination of PTFE resin and lubricant is then incubated in a warming cabinet overnight which is maintained at a temperature of approximately 85 degrees Fahrenheit (degrees F.). The incubation period is believed to allow for a further and more equal dispersion of the lubricant throughout the PTFE resin. If desired, further mixing and heating steps may be undertaken during the compounding process.

Next, the compounding of a suitable hydroxyapatite material is performed by first sifting the hydroxyapatite, through a suitable sieve (e.g., #40) using a mechanical sieve shaker. An amount of dry PTFE resin is then measured and added to the hydroxyapatite and is preferably performed in a room with air temperature below 70 degrees F. The hydroxyapatite and PTFE resin combination is shaken in a cold storage room for approximately three minutes. The hydroxyapatite and PTFE resin combination is then passed back into the cold room, and a lubricant is added to the composition. The resulting combination (hydroxyapatite+PTFE resin+lubricant) is shaken and then sifted through a number twenty (#20) sieve.

The combination is then incubated overnight in a warming cabinet which is maintained at an air temperature of approximately 85 degrees F., and removed from the incubator at least twenty minutes prior to pre-forming the mixture which is described below. The combination is then shaken and subsequently sifted through a number twenty (#20) sieve.

Following compounding, both the polymeric compound and the hydroxyapatite are pre-formed into a compressed cylinder by series of process steps. First, the hydroxyapatite compound is poured into the first area 48 of a divided pre-former by directing it through a funnel which is fit to the outside of the inner barrel. FIG. 2 illustrates the divided pre-form barrel 40 which is used in pre-forming the compounds into a compressed cylinder. The divided pre-form barrel 40 comprises an outer hollow cylindrical member 42, an inner hollow cylindrical member 44, and a central solid cylindrical member 46. The inner hollow cylindrical member 44 is concentrically contained within the outer hollow cylindrical member 42. Details of a similar process are shown and described in U.S. Pat. Nos. 5,827,327; 5,641,443; and 6,190,590, each of which are incorporated herein by reference.

The hydroxyapatite compound is poured within a first area 48 located between the inner hollow cylindrical member 44 and the central solid cylindrical member 46. The polymeric compound is then poured within a second area 52 located between the outer hollow cylindrical member 42 and the inner hollow cylindrical member 44.

In one of the preferred embodiments, the outer hollow cylindrical member 42 has a radius greater than the radius of the inner hollow cylindrical member 44. The diameter of the components which constitute the pre-form barrel will vary depending on the size and type of graft that is being produced. The pre-form barrel 40 that was used with the composition parameters set out in Example 1 had a radius of approximately 1.5 inches. The first area 48 between the inner hollow cylindrical member 44 and the central solid cylindrical member 46 had a radius of approximately 0.38 inches, the inner hollow cylindrical member 44 had a wall thickness of approximately 0.07 inches, and the second area 50 located between the outer hollow cylindrical member 42 and the inner hollow cylindrical member 44 had a radius of approximately 0.6 inches.

The materials contained in the divided pre-form barrel 40, namely the polymeric compound and the hydroxyapatite compound, are then compressed. The materials are compressed by placing the divided pre-form barrel 40 on a suitable press such as, for example, that shown in U.S. Pat. No. 5,827,327. After compressing the materials contained within the divided pre-form barrel 40, the inner cylindrical member 44, the outer cylindrical member 42, and the center solid cylindrical member 46 of the divided pre-form barrel 40 are removed to obtain a compressed cylinder of material. The inner hollow cylindrical member 44 is removed without disturbing the interface between the hydroxyapatite (or hydroxyapatite polymeric compound) and the polymeric compound.

The press used during the compression of the polymeric compound and the hydroxyapatite (or hydroxyapatite polymeric compound) is driven by a suitable power drive, which forces a top member toward a bottom member to compress the material within the divided pre-form barrel 40. Hollow cylindrical tubes of varying thicknesses are used to compress the material within the divided pre-form barrel 40 by slidably reciprocating around the inner hollow cylindrical member 44, the outer hollow cylindrical member 42, and the center solid cylindrical member 46 of the divided pre-form barrel 40. Alternatively, the dividers within the pre-form barrel may be removed prior to compression, without disturbing the interface between the different compounds, and then compressed to form a billet for extrusion. The compressed cylinder of material, or billet, is co-extruded via a suitable device such as, for example, the extruder shown in U.S. Pat. No. 5,827,327. Briefly, the compressed cylinder of material is placed within an extrusion barrel. Force is applied to a ram, which in turn expels pressure on the compressed cylinder of material. The pressure causes the compressed cylinder of material to be extruded around a mandrel, through extrusion die, and issue as a tubular extrudate. The tubular extrudate can be expanded to increase the porosity or alter the elasticity of the extrudate. After extrusion or expansion, the extrudate can be sintered in accordance with the expansion and sintering procedures undertaken with PTFE grafts which are known to those skilled in the art.

As another example, PTFE resin and other components were mixed as described herein and tabulated in Table 1 to provide for the graft device 100, which includes ePTFE layer 104 with first surface 104A and second surface 104B. In the preferred embodiments, hydroxyapatite or HA is coupled to the first surface 104A as HA portion 102; HA is also provided as an elongated portion 106 or 108 on the second surface 104B. Alternatively, silver chloride or tantalum powder can be provided for portion 108 with HA portion 106. Additionally, other suitable materials can be utilized in combination with HA such as, for example, gold, titanium, barium sulfate.

In this example, each mixture was labeled and a billet was formed using HA for portion 102 of the graft device 100. The pellet was extruded through a suitable extruder at a pressure from about 500 to about 2000 psi. The reduction ratio (i.e., wall thickness of billet to extruded graft thickness) for the billet can be from about 50 to about 350. The expansion ratio can be, as set forth in Table 2 below:

TABLE 1

| | | PTFE resin | HA | AgCl | Lube |
|---|---|---|---|---|---|
| Ref # | Formulation | wt. (g) | wt. (g) | wt. (g) | wt. (g) |
| 102 | HA luminal | 200 | 50 | — | 60 |
| | layer | | | | |
| 106 | HA line | 6 | 4 | — | 3.6 |
| 108 | Silver line | 6 | — | 4 | 3.6 |

TABLE 2

| Expansion Ratio | Starting length (cm) | Final length (cm) |
|---|---|---|
| 6 | 17 | 100 |
| 4 | 25 | 100 |
| 2 | 50 | 100 |

In this example, the billets were extruded to form various 6 millimeters tubes. Each extruded tube was then expanded to various lengths to introduce different degrees of porosity in the PTFE material, thereby providing the expanded PTFE or ePTFE. The expanded tubes were then sintered at a suitable sintering temperature to cause the tube to maintain essentially the desired porosity and improve the physical characteristics of the expanded ePTFE. The sintering temperature can be similar to that of standard ePTFE graft processing, which can be from about 200 degrees Fahrenheit to 400 degrees Fahrenheit, and preferably about 300 degrees Fahrenheit. As formed, the graft device 100 had a luminal layer and an orientation line containing HA. The graft device 100 also had silver chloride in the orientation line to improve visibility in a suitable imaging technique (e.g., x-ray imaging) and to release silver ions upon implantation so as to provide for anti-microbial characteristics. Further, the HA and silver chloride can be provided at any suitable locations on or in the graft device 100, as shown exemplarily in FIG. 1.

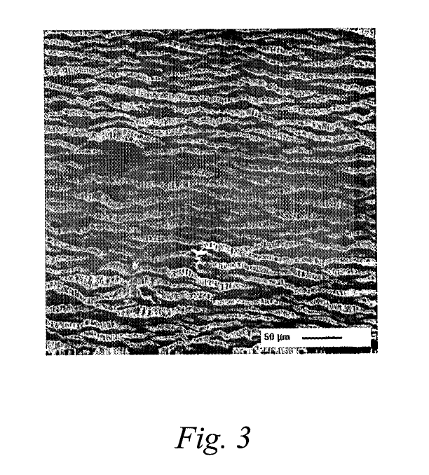

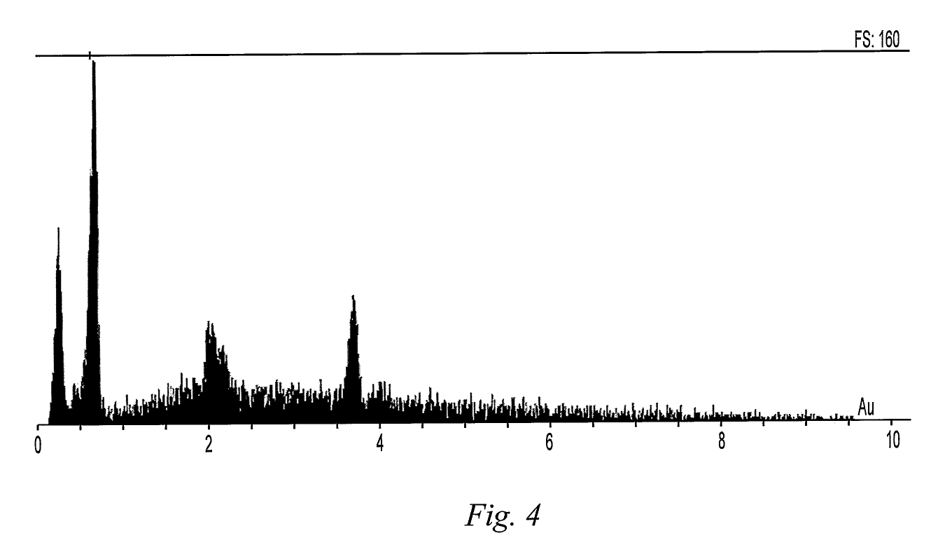

FIG. 3 shows scanning electron microscope image of the graft device 100 having HA coupled thereto. FIG. 3 shows that the nodes-fibril structure of the graft device 100 is similar to known ePTFE graft. However, FIG. 4 shows, in an Energy-Dispersive-X-ray (EDX) analysis of HA of the graft device 100, the presence of calcium and phosphorus, thereby confirming the presence of HA in the ePTFE.100301 Other techniques to provide for the graft device 100 are shown and described in U.S. Pat. Nos. 5,628,786; 6,053,943; 6,203,735 and U.S. Patent Application Publication Nos. 2004/0164445; 2004/0232588; 2004/0236400, each of which are incorporated herein by reference.

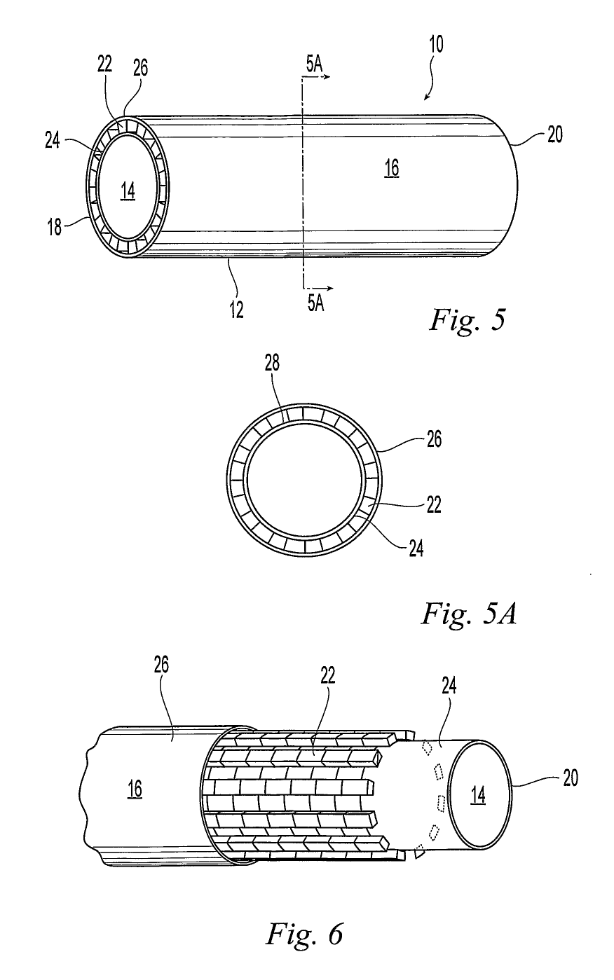

Referring now to FIGS. 5, 5A, and 6, an encapsulated stent or “stent-graft” 10 having the graft device 100 coupled to a support member 22, such as a stent, is shown in FIG. 5. The stent-graft 10 generally includes a tubular member 12 having an interior surface 14 and an exterior surface 16 which are contained between first and second ends 18, 20. As illustrated in FIGS. 5 and 6, the tubular member 12 includes a balloon or pressure expandable tubular shaped support member 22 which is loaded over a first biocompatible flexible tubular member 24 that is held on a mandrel (not shown). A second biocompatible flexible tubular member 26 is then loaded over the first biocompatible tubular member/support member combination.

The tubular shaped support member 22 preferably includes a stent similar to that described in U.S. Pat. Nos. 4,733,665; 6,053,941; 6,053,943; 5,707,386; 5,716,393; 5,860,999; 6,214,839, which are hereby incorporated by reference as if set forth in their entireties herein. The stent utilized for the member 22 can be balloon expandable stent, self-expanding stent or memory-shaped plastic stent.

The first and second biocompatible flexible tubular members 24, 26 are preferably made of expanded polytetrafluoroethylene (ePTFE) with hydroxyapatite coupled to the ePTFE, as described above. The first and second biocompatible flexible tubular members 24, 26 may also be made of unexpanded polytetrafluoroethylene (which can also be provided with hydroxyapatite, as described above. Further, the pressure expandable tubular shaped support member 22 may be made of any material having the strength and elasticity to permit radial expansion and resist radial collapse such as silver, titanium, stainless steel, gold, nickel-Titanium alloy, Nitinol, and any suitable plastic material capable of maintaining its shape and material properties at various sintering temperatures for PTFE or ePTFE.

A cross-sectional view of the stent-graft 10 is shown in FIG. 5A. The section plane is indicated as 5A-5A in FIG. 5. The cross-section view of FIG. 5A shows the stent-graft prior to fusing the graft members and prior to expansion. The first biocompatible flexible tubular member 24, preferably made of unsintered ePTFE, forms the innermost layer or luminal surface of the stent-graft 10, and covers the lumen 28 of the stent-graft 10, thereby providing a smooth, inert biocompatible blood flow surface. The tubular support member 22, preferably a stent or similarly constructed structure, forms the middle layer located at the center of the stent-graft 10. Finally, the second biocompatible flexible tubular member 26, which is also preferably made of unsintered ePTFE, forms the outermost layer or abluminal surface of the stent-graft 10.

After loading the tubular shaped members onto one another, pressure is applied to the graft/stent/graft assembly in order to fuse the first and second biocompatible flexible tubular members 24, 26 to one another through the openings contained within the tubular support member 22. Where the tubular support member 22 is a stent frame, the first and second ePTFE tubular members 24, 26 are fused to one another through the openings between the struts of the stent. The graft/stent/graft assembly is then heated at sintering temperatures to form a physical bond between the ePTFE layers. The resulting prosthesis is an unexpanded stent encapsulated within ePTFE layers, or specifically, an unexpanded stent having ePTFE and hydroxyapatite layers on its luminal surface and the stent and ePTFE layers are inseparable. Alternatively, the prosthesis can include hydroxyapatite on both its luminal and abluminal surfaces. Further, the ePTFE layers may also be fused or joined together around the ends of the unexpanded stent thereby entirely encasing the stent within ePTFE in both the radial and longitudinal directions. The resulting stent-graft can be loaded onto a suitable delivery device such as, for example, U.S. Pat. No. 6,756,007, which is incorporated herein by reference in its entirety.

Hydroxyapatite may be integrated in the tubular members 24, 26 as part of the two-layer extrusion structure described with reference to FIGS. 1-4, above. For example, the inner tubular member 24 may be a two-layer extrusion and applied to the support member 22 with the combined hydroxyapatite and ePTFE admixture layer on the lumen side (facing away from the support member 22) or ablumen side (facing toward the support member 22). Alternatively, or in addition, the outer tubular member 26 may be a two-layer extrusion and applied to the support member 22 with the combined hydroxyapatite and ePTFE admixture layer on the lumen side (facing toward the support member 22) or ablumen side (facing away from the support member 22). Another alternative is to form either or both of the inner tubular member 24 and/or the outer tubular member 26 of a monolithic hydroxyapatite and ePTFE admixture layer and apply one or both to the support member 22 as discussed above. Other combinations are possible as well, as discussed below.

Table 3 illustrates wall sections of some embodiments, going from the outer layer to the inner layer (lumen), using [HA-P] to denote hydroxyapatite and PTFE admixture, [P] to denote just PTFE, and [SM] to denote the support member.

TABLE 3
Embodiments of Grafts

| | | | |
|---|---|---|---|
| 1. | [SM] [HA] | 6. | [HA] [P] [SM] [HA] |
| 2. | [HA] [SM] | 7. | [HA] [SM] [P] [HA] |
| 3. | [HA] [SM] [HA] | 8. | [HA] [SM] [HA] [P] |
| 4. | [P] [SM] [HA] [P] | 9. | [P] [HA] [SM] [HA] [P] |
| 5. | [P] [HA] [SM] [HA] | 10. | [HA] [P] [SM] [P] [HA] |

The stent-graft may advantageously be used in a variety of medical applications including intravascular treatment of stenoses, aneurysms or fistulas; maintaining openings in the urinary, biliary, tracheobronchial, esophageal, renal tracts, vena cava filters; repairing abdominal aortic aneurysms; or repairing or shunting damaged or diseased organs such as, for example, Transjugular Intrahepatic Portosystemic Shunt (TIPS).

A TIPS is formed by an intrahepatic shunt connection between the portal venous system and the hepatic vein for prophylaxis of variceal bleeding, in the treatment of portal hypertension and its complications. Portal hypertension is believed to cause blood flow to be forced backward, causing veins to enlarge, resulting in variceal bleeding. In a typical TIPS procedure, a percutaneously created connection is provided by an implant within the liver between the portal and systemic circulations. Although this procedure has emerged as a less invasive alternative to surgery by reducing pressure gradient between portal and systemic circulations, there can be complications associated with the placement of the implant across the intrahepatic tract. Specifically, where a stent-graft with a bare stent portion (i.e., a “hybrid” stent graft having an uncovered stent portion coupled to a stent-graft or covered portion) is utilized in the procedure, it can become necessary to determine where the covered portion ends during the procedure in order to allow blood flow through the uncovered stent portion. Where the graft device 100 is configured as a hybrid stent-graft (not shown), the provision of HA provides for radio-opacity, which is believed to be advantageous in TIPS procedure. Thus, by virtue of the HA provided on the covered portion of the stent, a medical practitioner is able to view the actual position of the covered and uncovered portion of the hybrid stent-graft to determine its placement during the procedure without occluding blood flow.

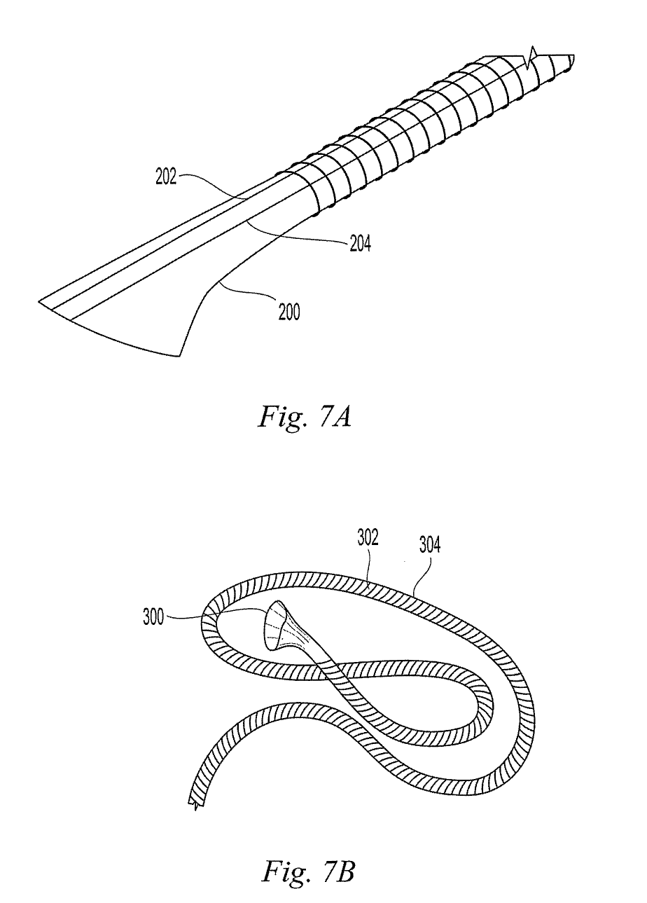

Referring to FIGS. 7A and 7B, hydroxyapatite can also be coupled to the material (or materials) forming the vascular bypass grafts 200 and 300. Vascular bypass graft 200 is configured for desired blood flow characteristics for applications above the knees, whereas bypass graft 300 is configured for blood flow characteristics below the knee. Regardless of the structural configurations and applications of the bypass grafts 200 and 300, HA can also be utilized with grafts 200 and 300 in a similar manner to the incorporation of HA with the graft device 100 described earlier. That is, HA can be incorporated with the synthetic non-metallic material (e.g., Dacron, polyester, PTFE, ePTFE, polyurethane, polyurethane-urea, siloxane, and combinations thereof) for grafts 200 and 300 with HA 202 (302 for graft 300) and silver chloride 204 (304 for graft 300) in at least one of the luminal and abluminal surfaces of the grafts 200 (300); dispersed through out the synthetic non-metallic material; coated thereon; spray coated thereon; dipped thereon; vapor deposited thereon; sputter-deposited thereon; or to form radio opaque surfaces on the grafts. The material or combinations of materials used (e.g., Dacron, polyester, PTFE, ePTFE, polyurethane, polyurethane-urea, siloxane, and combinations thereof) can include surface modifying additives or other materials. Examples of various grafts are shown and described in U.S. Pat. Nos. 6,203,735; 6,039,755; 6,790,226, each of which is incorporated in its entirety in this application.

Although the graft device 100 has been described in relation to specific examples noted above, it should be emphasized that variations in the configuration or composition of ePTFE, HA, stent framework, and other design parameters are to be utilized with the graft device 100. For example, the weight percentage of HA in the graft device can vary from 0.1 percent to 90 percent, and most preferably from 10 to 60 percent; the average HA particle size may range from about 20 nanometers to about 100 microns, and most preferably from 0.1 micron to 5 microns; the HA particle may be porous in certain configurations and non-porous in other configurations; the calcium to phosphorous atomic ratio within the HA can be in a range from about 1.2 to about 1.7 with a solid concentration of about 30% to about 70% by volume, and HA composition similar to the average composition of natural bone mineral is most preferred; HA may be obtained from natural sources such as natural bone material or may be obtained by synthetic processes; HA may be obtained as described in the preferred embodiments; other methods or techniques to couple HA on a suitable graft device can be utilized, such as, for example, sputtering, spraying, or low temperature deposition techniques; HA may constitute 100 percent of the luminal or abluminal surface of the graft device and can be homogeneously distributed thought the entire graft body; HA can also be coupled to the stent framework while HA is provided on at least one of the abluminal and luminal layers of the stent graft of the preferred embodiments; HA may constitute an adhesive film of about 10 microns to about 1000 microns film.

Furthermore, the HA particles may be used to carry biologically active compounds which may include but are not limited to compounds such as carbon particles, graphite particles, antibiotics (amethoprinrifampin or gentamycin); macrolide antibiotics; steroidal or anti inflammation agents (e.g., estradiol); antineoplastic agents; antifungals antivirals; antibodies; genetic sequence agents; growth factors inhibitors; angiogenesis; anti-angiogenesis; proteinase inhibitors; antiproliferative compounds or cell cycle modulators (such as rapamycin, sirolimus, or paclitaxel). Various methods or techniques known to those skilled in the art can be used to incorporate drugs or bioactive compounds in the HA. For example, drugs may be added after the HA-graft composite is made. Organic or aqueous solvent based techniques can be used to diffuse the drugs in the HA particles. Alternatively, HA particles may be first loaded with drugs and then incorporated in the graft. The drug may be released quickly within 60 minutes or can be released in a controlled manner from few days to two years. Additional polymeric coating or ceramic coating on HA particles may be used to control the release of the drug.

Additionally, where ePTFE is used in conjunction with HA, the composite HA ePTFE grafts may have different porosities and node-fibril structures. The preferred porosity or internodal distance may range from about 10 to about 40 micron range. Porosity of the ePTFE with about 5 microns to about 100 microns range may also be used. By controlling expansion ratios, lubricant levels, PTFE resin particle size and other ePTFE processing parameters, grafts with various porosities can be made to provide HA coupled grafts with regions of different porosities. The HA coupled graft may also be made using multiple layers of ePTFE graft tubes. The HA based grafts may also have additional features such as cuff to improve patency, beading to improve kink resistance, visible orientation lines to assist during implantation or other surgical procedures. Other ceramic materials such as nano-sized carbon tubes, calcium carbonate, and genetic or viral materials may also be used in conjunction with the HA material, which can be combined with at least one of the graft materials described herein. It should be noted that, as used herein, the term “HA” or “hydroxyapatite” is used to denote not only hydroxyapatite, but are used generically herein to define bio-compatible calcium salts, including but are not limited dicalcium phosphate, tricalcium phosphate, tetracalcium phosphate, and other compounds in the calcium phosphate or calcium carbonate family. Any of the members of the family of calcium salts described can be utilized as long as the salt is not substantially osteo-inductive (i.e., bone forming) in the graft device. As used herein, the singular form of “a,” “an,” and “the” include the plural referents unless specifically defined as only one. For example, the term “a calcium salt” is intended to mean either a single calcium salt or a combination of calcium salts.

Further methods and results are described below in which HA is incorporated in different concentrations into grafts.

### A Manufacturing

The manufacturing of the vascular grafts is divided into compounding, pre-forming, extrusion, crimping, drying, expansion, and sintering. The manufacturing is similar to carbon lined ePTFE graft wherein carbon is replaced with HA. In all grafts, HA is only added in the luminal layer.

1 Compounding

First, the PTFE resin and hydroxyapatite were sifted using a No. 20 sieve. Next, 500 grams oFirst, the PTFE resin and hydroxyapatite were sifted using a No. 20 sieve. Next, approximately 500 grams of PTFE resin was weighed into three jars. Lube dispensing level was corrected and 90 grams of lube was added to each jar. Each jar was shaken vigorously for about four minutes. All PTFE was then combined into one jar. For the HA portion of the grafts, about 250 grams of PTFE resin was weighed into 3 jars and 28, 63, or 128 grams of HA was added to each jar. This resulted in a weight/weight ratio of HA mixture in PTFE of approximately 10%, 20%, and 40%. Then approximately 50, 56, or 75 grams of lube was added into each jar. These jars were shaken vigorously by hand for 4 minutes. Finally, all jars were placed in the incubator at about 30° C. overnight.

2 Pre-Forming

The pre-forming involves forming the PTFE into a billet for extrusion. Molds used were same as used for Carboflo® carbon line ePTFE grafts. First, the jars were removed from the incubator and allowed to stabilize at room temperature for about 15 minutes. Before use, jars were shaken for about 15 seconds. Next, the barrel and plug assembly for the press was assembled. The HA-PTFE was sifted through a No. 20 sieve to remove any particles. The HA-PTFE was poured into the center of the funnel distributing the resin uniformly around the center shaft. Then, the billet was formed by compacting in the billet press. The pressure was between from 80 and to 85 psi. After compacting, the assembly was removed and the billet was extracted from the barrel. Finally, the billet was wrapped in aluminum foil for extrusion.

3 Extrusion

The extrusion converts billet into hollow tube (graft). First, the extrusion equipment was cleaned and assembled: the mandrel was screwed onto the center shaft, the billet was slid over the mandrel and extruder barrel, and the die was loaded over the mandrel. Next, the placement of the mandrel inside the die was checked using the extrusion depth gauge. The computer was set up with appropriate information and the extruder was set in the “Forward” position. As the pressure begins to increase, the extrudate started coming out of the die. Then, collection of the grafts started when the pressure stabilizes. The cutter cuts the graft at the appropriate length and the handler places the grafts on a lubed tray. Finally, after extrusion, all equipment was cleaned and number of extruded grafts was recorded.

4 Crimping

Crimping involves modification of grafts ends with metallic clips which helps in expansion of the graft. First, the appropriate crimp plates were obtained (6 RW at 33 cm). Next, five grafts were lined up along the grooves of the plate. Ends of the grafts were cut until even with the plate. Then, the rings were placed over the brass plugs and inserted into the ends of the graft. The rings were slid over the grafts until they fit over the grooves of the plate. The plate bars were placed over the rings. Finally, the entire plate assembly was placed under the billet press. The rings were compacted over the graft and plug.

5 Drying

This is necessary to remove all lube from the grafts. The grafts were dried in a large oven at about 40° C. for one hour.

6 Expansion

The expansion is required to manufacture expanded polytetrafluoroethylene. First, the grafts were placed on racks in the large oven. The expansion program on the computer controls the temperature and actual expansion. Next, the grafts undergo the expansion cycle. Finally, the grafts were removed and unattached from the expansion rack.

7 Sintering

Sintering the grafts strengthens mechanical properties such as tensile strength. First, the grafts were loaded onto the sintering rack. Next, the grafts were placed into the large oven. Then, the grafts were sintered for thirty seconds at 360° C. Finally, the grafts were removed and the plugs were cut off.

### B Testing

The vascular grafts produced were tested for suture retention strength, radial tensile strength, and longitudinal tensile strength. These physical dimensions were measured: morphology, internodal distance, chemical composition, inside diameter, and wall thickness. The strength tests were performed on the Instron 5500 series with a 10 lb and 400 lb load cell. The physical characteristics were determined by light microscopy and EDX-SEM.

### C Scanning Electron Microscopy (SEM)

SEM was performed with inner, outer, radial, and longitudinal sections taken randomly from each graft. Each sample was coated with iridium two times to ensure proper dispersion of the conducting material. The magnifications used to analyze the samples were 100×, 500×, and 1000×; the images were taken at 10 keV. Energy dispersive x-ray analysis (EDX) was used in conjunction with SEM in order to determine the chemical composition of the samples. EDX determination was done at 20 keV.

### Results

Table 4 summarizes the physical and mechanical properties of HA coated vascular grafts. The average values were shown. The 20% graft only yielded a few grafts and one was submitted for testing by quality control.

TABLE 4
Average mechanical properties of HA coated vascular grafts.

| | Wall | Inner | | Radial | Longitudinal | Internodal | Water Entry | Suture Retention |
|---|---|---|---|---|---|---|---|---|
| | Thickness | Diameter | Eccentricty | Tensile Strength | Tensile Strength | Distance | Pressure | Strength |
| Graft | (mm) | (mm) | (%) | (psi) | (gF/mm²) | (μm) | (psi) | (gf) |
| Control | 0.682 | 5.83 | 4 | 798 | 2102 | 15 | 9.5 | 198 |
| 10% HA | 0.692 | 6 | 5.6 | 536 | 1700 | 24.6 | 7 | 208 |
| 20% HA | 0.683 | 6.1 | 7 | 520 | 2009 | 23 | 8.8 | 344 |
| 40% HA | 0.711 | 5.8 | 6 | 436 | 1865 | 19.6 | 7.7 | 440 |

It can be seen that the mechanical properties of the grafts are slightly affected by the addition of HA, but are still within normal range of typical ePTFE graft. Increased HA resulted into higher reduction in radial and longitudinal strength. However, the decrease is not sufficient to affect performance of the vascular graft. Internodal distance is within the standard tolerance of the standard ePTFE graft product (10 to 40 microns). Suture strength of the graft increased with increasing HA percentage. This may be due to “volume filling effect” of HA material in the porosity of the graft.

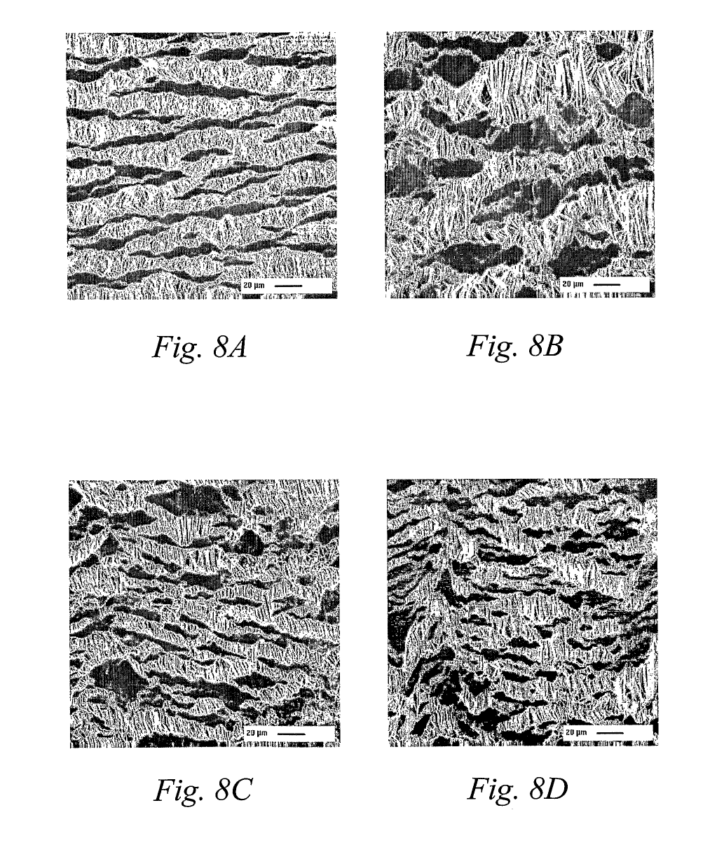

The samples required coating with iridium two times to ensure proper coating was achieved. However, in some images, white streaks are still apparent. The white streaks seen in the images are a result of charging on the sample. PTFE is a nonconductive material and must be coated with a conductive metal for imaging. Insufficient coating of the sample will result in collection of charge on the sample and streaky images. FIGS. 8A through 8D depict the sample inner sections at 500×. The outer sections of the grafts did not differ from the control graft morphology. The fibers looked similar and the internodal space was consistent.

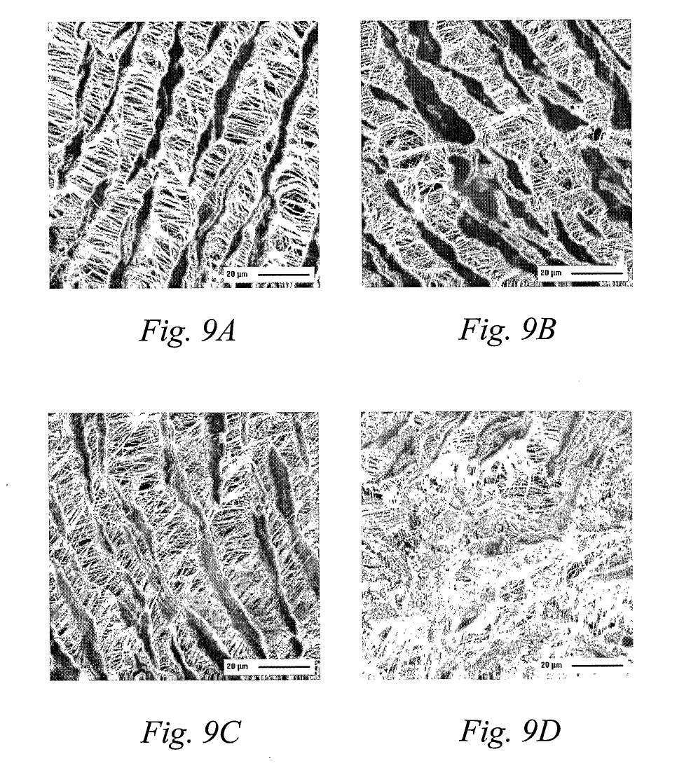

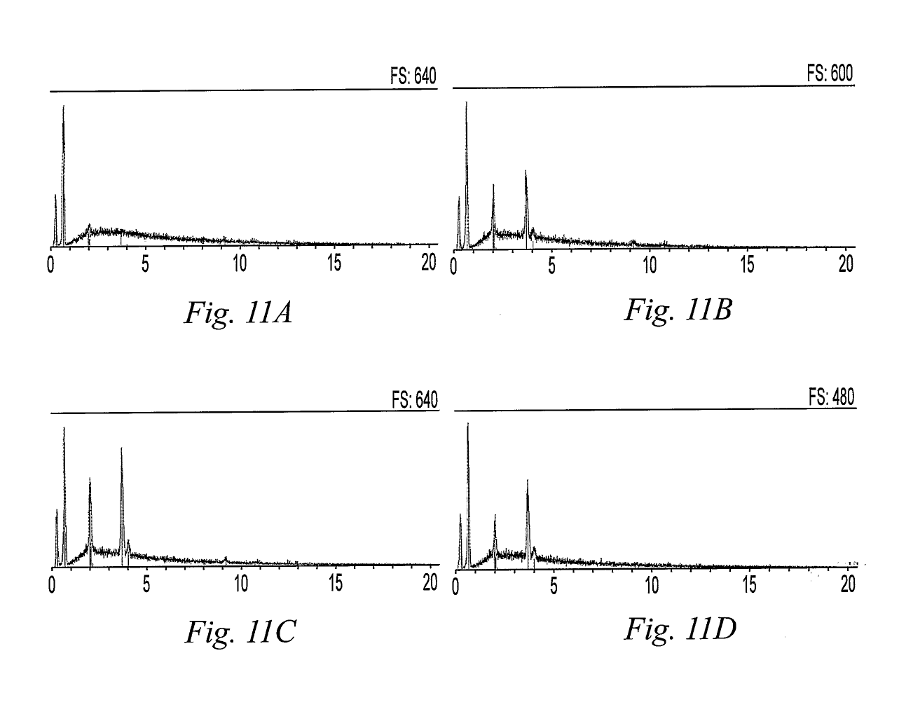

FIGS. 9A through 9D depict the vascular grafts along a longitudinal cut. Images were taken at 1000× along the edge to better see the hydroxyapatite. FIGS. 11A through 11D are images of the radial sections of the grafts taken at 1000×.

FIGS. 12A through 12D are graphs from EDX analysis. The chemical formula of hydroxyapatite is Ca₁₀(PO₄)₆(OH)₂. Therefore, the presence of calcium and phosphorus in the EDX spectrum will show the successful incorporation of HA. An iridium peak shows up in some spectra; Iridium was used to coat the ePTFE grafts to yield conductivity.

The inner section of the HA grafts have been modified to include hydroxyapatite. It is expected to have P and Ca peaks as well as the C and F peaks from the PTFE. All the inner sections of the HA grafts have P and Ca detected.

The addition of hydroxyapatite may be beneficial to the long term patency of vascular grafts. Since hydroxyapatite is a natural ceramic found in bone, the hemocompatibility of a new composite vascular graft is likely to be improved. Using established manufacturing practices for the incorporation of carbon into the luminal layer of ePTFE vascular grafts, three different compositions of hydroxyapatite grafts were manufactured. For the inner layer, 10% w/w, 20% w/w, and 40% w/w of hydroxyapatite was added to PTFE. It was possible to accomplish the manufacturing: compounding, pre-forming, extrusion, crimping, drying, expansion, and sintering. A control graft of containing only PTFE and no HA was also made.

All mixtures were incubated at least overnight before pre-forming. It was found to be helpful, in some cases, if the center is pulled slowly with agitation of the bar (e.g., hitting lightly with hammer). The overall the extrusion pressures were as follows. For the 10% HA mixture, the pressure was around 1300 psi while the other mixtures were around 1100 psi. The extrusion for the 40% HA mixture produced grafts that were noticeably lumpy. After expansion and sintering of the 40% grafts, there were grafts with defects in which small parts of the grafts were thinner.

Following manufacturing, mechanical testing on the grafts was performed to determine if the behavior of the new grafts differed from standard grafts. As the percentage of HA is increased, the radial tensile strength tend to be decreased when compared with the control graft. The only aberration is in the suture retention strength data. The increase of suture retention strength with higher concentrations of hydroxyapatite could result from the resistance against longitudinal pull by the hydroxyapatite particles.

SEM imaging required coating the grafts twice with iridium in order to obtain enough conductivity off the surface of the sample. The images were taken at 10 keV and the EDX samples were taken at 20 keV. Uneven coating on some samples led to streaking on the images. The SEM images demonstrated that the hydroxyapatite was retained in the inner portion of the vascular grafts. There was a difference between the control grafts and the HA grafts in the fiber shape and presence of particles. The fibers of the control graft seem more elongated and regular. It was hard to distinguish where the hydroxyapatite layer began; however, it seems that as the concentration of HA increased, the more pronounced and thicker the layer appeared. Overall, the pictures demonstrated the presence of HA in the new grafts.

Ability of graft to support endothelial, fibroblast cells and smooth muscle cells is being tested. It is expected that the HA will improve attachment of endothelial cells.

It was found to be possible to extrude different concentrations of hydroxyapatite in the ePTFE graft. From a billet, an average of 30 grafts can be obtained. Based on the mechanical data and experience in manufacturing, the 10% HA graft appears to be the best candidate from a manufacturing point of view. It retained the most physical characteristics of a control ePTFE graft and was easiest to extrude. The highest concentration of HA (40%) had defects during manufacturing and had discrepancies in the final product, but for some applications, it may be a preferred concentration.

While the present invention has been disclosed with reference to certain preferred embodiments, numerous modifications, alterations, and changes to the described embodiments are possible without departing from the sphere and scope of the present invention, as defined in the appended claims. Moreover, where methods, processes described above indicate that certain events occurring in certain order, those skilled in the art would recognize that the ordering of steps may be modified and that such modifications are within the variations of the described embodiments. Accordingly, it is intended that the present invention not be limited to the described embodiments, but that it have the full scope defined by the language of the following claims, and equivalents thereof.

1. A graft device comprising:
a layer of synthetic non-metallic material having a first surface and a second surface spaced apart from the first surface; and
an inorganic bio-compatible calcium salt coupled to at least one of the first and second surfaces of the synthetic non-metallic material.

2. The graft device according to claim 1, wherein the synthetic non-metallic material comprises a material selected from a group consisting essentially of Dacron, polyester, PTFE, ePTFE, polyurethane, polyurethane-urea, siloxane, and combinations thereof.

3. The graft device according to claim 1, wherein the synthetic non-metallic material comprises ePTFE having internodal distance of about 10 microns to about 40 microns and a porosity of about 5 microns to about 100 microns.

4. The graft device according to claim 2, wherein the layer of ePTFE comprises an average thickness of about 40 to 300 microns.

5. The graft device according to claim 1, wherein the synthetic non-metallic material comprises ePTFE and the bio-compatible calcium salt comprises hydroxyapatite having particles with an average size of about 20 nanometers to about 100 microns.

6. The graft device according to claim 1, wherein the inorganic bio-compatible calcium salt comprises a calcium to phosphorus ratio from about 1.2 to about 1.7.

7. The graft device according to claim 1, wherein the inorganic bio-compatible calcium salt comprises porous hydroxyapatite coupled to at least one biologically active agent.

8. The graft device according to claim 7, wherein the at least one biologically active agent is selected from a group consisting essentially of antibiotics, anti-renosis agents, anti-proliferative agents, and combinations thereof.

9. The graft device according to claim 8, wherein the anti-restenosis agents comprise one of paclitaxel and rapamycin.

10. The graft device according to claim 9, wherein at least one of the ePTFE layer and the hydroxyapatite includes a layer of silver chloride.

11. The graft device according to claim 2, further comprising a stent frame work having a portion of the frame work encapsulated by the synthetic non-metallic material.

12. The graft device according claim 1 further comprising a flared end portion defining a generally elliptical perimeter being coupled to the graft device.

13. The graft device of claim 1, wherein the inorganic bio-compatible calcium salt is impregnated with the synthetic non-metallic material.

14. The graft device of claim 1, wherein the inorganic biocompatible calcium salt is encapsulated in the synthetic non-metallic material.

15. The graft device of claim 1, wherein the inorganic biocompatible calcium salt is encapsulated by the synthetic non-metallic material.

16. An implant device comprising:
a stent frame;
a synthetic non-metallic material that surrounds a portion of the stent frame, the synthetic non-metallic material having first and second surfaces; and
an inorganic bio-compatible calcium salt coupled to at least one of the first and second surfaces of the synthetic non-metallic material.

17. The implant device according to claim 16, wherein the synthetic non-metallic material comprises a material selected from a group consisting essentially of Dacron, polyester, PTFE, ePTFE, polyurethane, polyurethane-urea, siloxane, and combinations thereof.

18. The implant device according to claim 16, wherein the synthetic non-metallic material comprises ePTFE having internodal distance of about 10 microns to about 40 microns.

19. The implant device according to claim 16, wherein the ePTFE comprises a plurality of layers of ePTFE.

20. The implant device according to claim 1, wherein the layer of ePTFE comprises an average thickness of about 40 to 300 microns.

21. A method of endothiealizing a graft comprising:
coupling a synthetic non-metallic material with inorganic bio-compatible calcium salt to form a composite graft device; and
implanting the composite graft device in body vessel of a mammal.

22. The method of claim 21, wherein the coupling comprises sputtering the inorganic bio compatible calcium salt on at least one surface of the synthetic non-metallic material.

23. The method of claim 21, wherein the coupling comprises spraying the inorganic bio compatible calcium salt on at least one surface of synthetic non-metallic material.

24. The method of claim 23, wherein the coupling comprises providing ePTFE.

25. The method of claim 24, wherein the coupling comprises extruding the inorganic bio compatible calcium salt as a layer with at least one layer of ePTFE to form a tubular member having a first length.

26. The method of claim 25, wherein the extruding comprises expanding the tubular member to about 50% of the first length.

27. The method of claim 20, wherein the expanding comprises sintering the tubular member.

28. A method of making a composite graft comprising:
providing a non-metallic material;
providing inorganic bio-compatible calcium salt; and
coupling inorganic bio-compatible calcium salt to the non-metallic material.

29. The method of claim 28, wherein the non-metallic material comprises a synthetic fiber.

30. The method of claim 29, wherein the synthetic fiber is selected from a group of material consisting essentially of Dacron, polyester, PTFE, ePTFE, polyurethane, polyurethane-urea, siloxane, and combinations thereof.

31. The method of claim 28, wherein the coupling comprises extruding the PTFE and hydroxyapatite.

32. The method of claim 28, wherein the coupling comprises forming at least one layer of PTFE coupled to at least one layer of hydroxyapatite.

33. The method of claim 28, wherein the extruding comprises expanding the PTFE to provide for expanded PTFE.

34. The method of claim 28, further comprising sintering the PTFE and hydroxyapatite.

35. The method of claim 28, wherein the non-metallic material comprises ePTFE having internodal distance of about 10 microns to about 40 microns and a porosity of about 5 microns to about 100 microns.

36. The graft device of claim 28, wherein the layer of ePTFE comprises an average thickness of about 40 to 300 microns.

37. The graft device of claim 28, wherein the non-metallic material comprises ePTFE and the hydroxyapatite includes particles having an average size of about 20 nanometers to about 100 microns.

38. The graft device of claim 28, wherein the hydroxyapatite comprises a calcium to phosphorus ratio from about 1.2 to about 1.7.

39. The graft device of claim 28, wherein the inorganic bio-compatible calcium salt comprises porous hydroxyapatite coupled to at least one biologically active agent.

40. The graft device according to claim 39, wherein the at least one biologically active agent is selected from a group consisting essentially of antibiotics, anti-renosis agents, anti proliferative agents, and combinations thereof.

41. A graft, comprising:
a first layer forming a first surface including an admixture of polymeric material and calcium salt;
a second layer including expanded polymeric material joined with the first layer.

42. A graft as in claim 41, wherein the polymeric material includes ePTFE having internodal distance of about 10 microns to about 41 microns and a porosity of about 5 microns to about 100 microns.

43. A graft as in claim 41, wherein the second layer has an average thickness of about 41 to 300 microns.

44. A graft as in claim 41, wherein the second layer is porous.

45. A graft as in claim 41, wherein the admixture is of polytetrafluoroethylene and hydroxyapatite.

46. A graft as in claim 41, wherein the first layer defines a lumen and the second layer surrounds the first layer.

47. A graft as in claim 41, wherein the first layer defines an annular flow channel.

48. A method of forming a graft, comprising:
forming a billet from an admixture of divided bio-compatible calcium salt and a divided non-metallic material;
extruding the billet.

49. The method as in claim 48, further comprising enveloping a stent with an extrudate formed by extruding the billet.

50. The method as in claim 49, wherein the calcium salt includes hydroxyapatite.

51. The method as in claim 48, wherein the forming includes mixing the calcium salt with a resin and a lubricant.

52. The method as in claim 48, wherein the non-metallic material includes polytetrafluoroethylene.

53. The method as in claim 52, wherein the calcium salt includes hydroxyapatite.

54. The method as in claim 48, wherein the billet includes an admixture layer of calcium salt mixed with polymeric material and an annular layer surrounding the admixture layer of polymeric material;
the extruding including coextruding the billet.

55. The method as in claim 54, wherein the extruding includes forming a tubular structure.

56. The method as in claim 55, further comprising expanding the extrudate resulting from the extruding.

57. The method as in claim 56, wherein the expanding includes sintering.

58. The method as in claim 48, further comprising expanding the extrudate resulting from the extruding.

59. The method as in claim 58, wherein the expanding includes sintering.
